(19) United States
(12) Patent Application Publication
(10) Pub. No.: US 2004/0157860 A1
(43) Pub. Date: Aug. 12, 2004

(54) Pyrazine compounds as CRF modulators

(76) Inventor: John W. Mickelson, Mattawan, MI (US)

Correspondence Address: PFIZER INC, 150 EAST 42ND STREET, 5TH FLOOR - STOP 49, NEW YORK, NY, 10017-5612, US

(21) Appl. No.: 10/706,555

(22) Filed: Nov. 12, 2003

Related U.S. Application Data
(60) Provisional application No. 60/428,146, filed on Nov. 21, 2002.

(51) Int. Cl. A61K 31/497; A61K 31/4965; C 07D 4 3/02

(52) U.S. Cl. 514/255.05; 544/405

(57) ABSTRACT

Disclosed are pyrazine derivatives, pharmaceutical compositions containing them, and methods of using them to treat a disorder or condition the treatment of which can be effected or facilitated by antagonizing a CRF receptor, such as anxiety disorders.

### CROSS-REFERENCE TO RELATED APPLICATIONS

This application claims the benefit of U.S. Provisional Application Serial No. 60/428,146 filed on 21 Nov. 2002.

### FIELD OF THE INVENTION

This invention relates to pyrazine derivatives, pharmaceutical compositions containing them, and methods of using them to treat a disorder or condition which can be effected of facilitated by antagonizing a CRF receptor, such as of anxiety disorders, depression and stress related disorders.

### BACKGROUND OF THE INVENTION

Corticotropin releasing factor (CRF) is a 41 amino acid peptide that is the primary physiological regulator of proopiomelanocortin (POMC) derived peptide secretion from the anterior pituitary gland [J. Rivier et al., Proc. Natl. Acad. Sci (USA) 80:4851 (1983); W. Vale et al., Science 213:1394 (1981)]. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in the brain [W. Vale et al., Rec. Prog. Horm. Res. 39:245 (1983); F. Koob, Persp. Behav. Med. 2:39 (1985); E. B. De Souza et al., J. Neurosci. 5:3189 (1985)]. There is also evidence that CRF plays a significant role in integrating the response in the immune system to physiological, psychological, and immunological stressors [J. E. Blalock, Physiological Reviews 69:1 (1989); J. E. Morley, Life Sci. 41:527 (1987)].

There is evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis, as they relate to the dysfunction of CRF neurons in the central nervous system [for a review, see: E. B. De Souze, Hosp. Practice 23:59 (1988)].

Anxiety disorders are a group of diseases, recognized in the art, that includes phobic disorders, anxiety states, post-traumatic stress disorder and atypical anxiety disorders [The Merck Manual of Diagnosis and Therapy, 16^{th} edition (1992)]. Emotional stress is often a precipitating factor in anxiety disorders, and such disorders generally respond to medications that lower response to stress.

In affective disorder, or major depression, the concentration of CRF is significantly increased in the cerebral spinal fluid (CSF) of drug-free individuals [C. B. Nemeroff et al., Science 226:1342 (1984); C. M. Banki et al., Am. J. Psychiatry 144:873 (1987); R. D. France et al., Biol. Psychiatry 28:86 (1988); M. Arato et al., Biol. Psychiatry 25:355 (1989)]. Furthermore, the density of CRF receptors is significantly decreased in the frontal cortex of suicide victims, consistent with a hypersecretion of CRF [C. B. Memeroff et al., Arch. Gen. Psychiatry 45:577 (1988)]. In addition, there is a blunted adrenocorticotropin (ACTH) response to CRF (i.v. administered) observed in depressed patients [P. W. Gold et al., Am. J. Psychiatry 141:619 (1984); F. Holsboer et al., Psychoneuroendocrinology 9:147 (1984); P. W. Gold et al., New Engl. J. Med. 314:1129 (1986)]. Preclinical studies in rats and non-human primates provide additional support for the hypothesis that hypersecretion of CRF may be involved in the symptoms seen in human depression [R. M. Sapolsky, Arch. Gen. Psychiatry 46:1047 (1989)]. There is also preliminary evidence that tricyclic antidepressants can alter CRF levels and thus modulate the numbers of receptors in the brain [Grigoriadis et al., Neuropsychopharmacology 2:53 (1989)].

CRF has also been implicated in the etiology of anxiety-related disorders, and is known to produce anxiogenic effects in animals. Interactions between benzodiazepine/non-benzodiazepine anxiolytics and CRF have been demonstrated in a variety of behavioral anxiety models [D. R. Britton et al., Life Sci. 31:363 (1982); C. W. Berridge and A. J. Dunn Regul. Peptides 16:83 (1986)]. Preliminary studies using the putative CRF receptor antagonist α-helical ovine CRF (9-41) in a variety of behavioral paradigms demonstrates that the antagonist produces “anxiolytic-like” effects that are qualitatively similar to the benzodiazepines [C. W. Berridge and A. J. Dunn Horm. Behav. 21:393 (1987), Brain Research Reviews 15:71 (1990)].

Neurochemical, endocrine and receptor binding studies have all demonstrated interactions between CRF and benzodiazepine anxiolytics, providing further evidence for the involvement of CRF in these disorders. Chlodiazepoxide attenuates the “anxiogenic” effects of CRF both in the conflict test [K. T. Britton et al., Psychopharmacology 86:170 (1985); K. T. Britton et al., Psychopharmacology 94:306 (1988)] and in the acoustic startle test [N. R. Swerdlow et al., Psychopharmacology 88:147 (1986)] in rats. The benzodiazepine receptor antagonist Ro 15-1788, which was without behavioral activity alone in the operant conflict test, reversed the effects of CRF in a dose-dependent manner while the benzodiazepine inverse agonist FG 7142 enhanced the actions of CRF [K. T. Britton et al., Psychopharmacology 94:396 (1988)]. The mechanisms and sites of action through which conventional anxiolytics and antidepressants produce their therapeutic effects remain to be elucidated. Preliminary studies, examining the effects of a CRF₁ receptor antagonist peptide (α-helical CRF₉₋₄₁) in a variety of behavioral paradigms, have demonstrated that the CRF₁ antagonist produces “anxiolytic-like” effects qualitatively similar to the benzodiazepines [for a review, see: G. F. Koob and K. T. Britton, In: Corticotropin-Releasing Factor: Basic and Clinical Studies of a Neuropeptide, E. B. De Souza and C. B. Nemeroff eds., CRC Press p. 221 (1990)].

The use of CRF₁ antagonists for the treatment of Syndrome X has also been described in U.S. patent application Ser. No. 09/696,822, filed Oct. 26, 2000, and European Patent Application No. 003094414, filed Oct. 26, 2000, which are also incorporated in their entireties herein by reference. Methods for using CRF₁ antagonists to treat congestive heart failure are described in U.S. Ser. No. 09/248,073, filed Feb. 10, 1999, now U.S. Pat. No. 6,043,260 (Mar. 28, 2000) which is also incorporated herein in its entirety by reference.

CRF is known to have a broad extrahypothalmic distribution in the CNS, contributing therein to a wide spectrum of autonomic behavioral and physiological effects [see, e.g., Vale et al., 1983; Koob, 985; and E. B. De Souze et al., 1985]. For example, CRF concentrations are significantly increased in the cerebral spinal fluid of patients afflicted with affective disorder or major depression [see, e.g., Nemeroff et al., 1984; Banki et al., 1987; France et al., 1988; Arato et al., 1989]. Moreover, excessive levels of CRF are known to produce anxiogenic effects in animal models [see, e.g., Britton et al., 1982; Berridge and Dunn, 1986 and 1987], and CRF₁ antagonists are known to produce anxiolytic effects; accordingly, therapeutically effective amounts of compounds provided herein are, for example, determined by assessing the anxiolytic effects of varying amounts of the compounds in such animal models.

The following patents or patent applications disclose compounds as antagonists of CRF₁ receptors: WO0160806, WO9735901, WO9829119, WO9736886, WO9736898, and U.S. Pat. Nos. 5,872,136, 5,880,140, and 5,883,105. The compounds are useful for treating CNS-related disorders, particularly affective disorders and acute and chronic neurological disorders. None of the above references, however, discloses the compounds of the present invention.

### SUMMARY OF THE INVENTION

We have found that compounds of Formula I, as well as stereoisomers thereof, pharmaceutically acceptable salts thereof, and prodrugs thereof, are CRF₁ antagonists and are useful in the treatment of disorders and diseases associated with CRF₁ receptors, including CNS-related disorders and diseases, particularly psychiatric disorders, affective disorders such as anxiety disorders, depression and stress related disorders, and acute and chronic neurological disorders and diseases. The compounds are also useful in smoking cessation programs.

Thus, in a first aspect, this invention provides a compound of Formula I, a stereoisomer thereof, a pharmaceutically acceptable salt thereof, or a prodrug thereof, which is useful as an antagonist of CRF₁ receptor, or as a treatment of disorders or diseases that are associated with CRF₁ receptors, or disorders the treatment of which can be effected or facilitated by antagonizing CRF, in a mammal, particularly in a human, such as social anxiety disorder; panic disorder; obsessive-compulsive disorder; anxiety with co-morbid depressive illness; affective disorder; anxiety; and depression.

In another aspect, the present invention provides for use of a compound of the invention for treating a disorder or disease that is associated with CRF₁ receptors, or a disorder the treatment of which can be effected or facilitated by antagonizing CRF, in a mammal, particularly in a human, such as social anxiety disorder; panic disorder; obsessive-compulsive disorder; anxiety with co-morbid depressive illness; affective disorder; anxiety; and depression.

In still another aspect, the present invention provides for a composition comprising a compound of the invention useful for treatment of a disorder disclosed herein above in a mammal, particularly in a human.

In still another aspect, the present invention provides for use of a compound of the invention in a binding assay, wherein one or more of the compounds may be joined to a label, where the label can directly or indirectly provide a detectable signal. Various labels include radioisotopes, fluorescers, chemiluminescers, specific binding molecules, particles, e.g. magnetic particles, and the like.

In yet another aspect, the present invention relates to the use of the compounds of the invention (particularly labeled compounds of this invention) as probes for the localization of receptors in cells and tissues and as standards and reagents for use in determining the receptor-binding characteristics of test compounds.

Labeled compounds of the invention may be used for in vitro studies such as autoradiography of tissue sections or for in vivo methods, e.g. PET or SPECT scanning. Particularly, compounds of the invention are useful as standards and reagents in determining the ability of a potential pharmaceutical to bind to the CRF₁ receptor.

### DETAILED DESCRIPTION OF THE INVENTION

In a first aspect, the present invention provides a compound of Formula I,

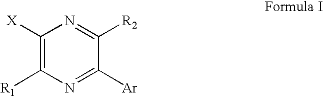

a stereoisomeric form thereof, a mixture of stereoisomeric forms thereof, a pharmaceutically acceptable salt thereof, or a prodrug thereof, wherein in formula I,

X is selected from a modified monocyclic group, aryl cycloalkyl, substituted aryl cycloalkyl, heteroaryl cycloalkyl, substituted heteroaryl cycloalkyl, aryl heterocycloalkyl, substituted aryl heterocycloalkyl, heteroaryl heterocycloalkyl, or substituted heteroaryl heterocycloalkyl (point of attachment being either nitrogen or carbon);

modified monocyclic group is selected from cycloalkyl, aryl, heterocycloalkyl, heteroaryl that is substituted with Y or (CR_{b}R_{b})ₙZ, wherein,

Y is selected from CN, NO₂, C(O)Rₐ, C(S)Rₐ, C(O)ORₐ, C(S)ORₐ, C(O)NRₐRₐ, C(S)NRₐRₐ, NRₐC(O)Rₐ, NRₐC(S)Rₐ, NRₐC(O)NRₐRₐ, NRₐC(S)NRₐRₐ, NRₐC(O)ORₐ, OC(O)Rₐ, OC(S)Rₐ, OC(O)NRₐRₐ, OC(S)NRₐRₐ, S(O)ₘNRₐRₐ, NRₐS(O)ₘRₐ, aryl, substituted aryl, heteroaryl, substituted heteroaryl, heterocycloalkyl, substituted heterocycloalkyl, cycloalkyl, substituted cycloalkyl, OR_{c}, and NHR_{c};

Z is selected from Y, ORₐ, NRₐRₐ, and S(O)ₘRₐ;

R_{b} is independently selected from H, alkyl, aryl, heteroaryl, heterocycloalkyl, or cycloalkyl optionally substituted with 1-5 Rt;

R_{c} is selected from aryl, heteroaryl, heterocycloalkyl, or cycloalkyl optionally substituted with 1 to 5 of Rt;

n is selected from 1-6; and

m is selected from 0, 1, and 2;

Ar is selected from aryl, substituted aryl, heteroaryl, substituted heteroaryl;

R₁, R₂, are independently selected from H, halogen, —NO₂, —CN, —ORₐ, —NRₐRₐ, —C(O)Rₐ, —C(O)NRₐRₐ, —C(S)NRₐRₐ, —C(O)ORₐ, —C(S)ORₐ, S(O)ₘRₐ, —S(O)ₘNRₐRₐ, —NRₐS(O)ₘRₐ, —NRₐC(O)ORₐ, —NRₐC(O)Rₐ, —NRₐC(O)NRₐRₐ, —NRₐC(S)NRₐRₐ, and —OC(O)NRₐRₐ, —OC(O)Rₐ, OC(O)ORₐ, CR_{b}R_{b}Z, R_{f};

Rₐ is independently selected from H, alkyl, cycloalkyl, haloalkyl, aryl, heteroaryl, or heterocycloalkyl optionally substituted with 1 to 5 of Rₜ, oxo (═O), thione (═S), phenyl, heteroaryl, or heterocycloalkyl where phenyl, heteroaryl, and heterocycloalkyl are optionally substituted with 1 to 5 independently taken from Rₜ;

R_{f} is independently selected from ethyl, propyl, butyl, pentyl, cycloalkyl, haloalkyl, aryl, heteroaryl, or heterocycloalkyl optionally substituted with 1 to 5 of Rₜ, oxo (═O), thione (═S), phenyl, heteroaryl, or heterocycloalkyl where phenyl, heteroaryl, and heterocycloalkyl are optionally substituted with 1 to 5 independently taken from Rₜ;

Rₜ is independently selected from R_{w}, halogen, —NO₂, —NR_{w}R_{w}, —OR_{w}, —SR_{w}, —CN, —C(O)NR_{w}R_{w}, —C(O)R_{w}, —OC(O)NR_{w}R_{w}, —OC(O)R_{w}, —NR_{w}C(O)R_{w}, —NR_{w}C(O)₂NR_{w}R_{w}, —NR_{w}C(O)OR_{w}, —S(O)ₘR_{w}R_{w}, —NR_{w}S(O)ₘR_{w}, —S(O)₂NR_{w}R_{w}, —NR_{w}S(O)₂NR_{w}R_{w}; and

R_{w} is independently selected from H, alkyl, cycloalkyl, phenyl, benzyl, heteroaryl or heterocycle where phenyl, benzyl, heteroaryl and heterocycloalkyl may be optionally substituted with alkyl or halogen.

Preferred compounds of the invention include:

compounds of Formula I wherein, in Formula I, X is a modified monocyclic group;

compounds of Formula I wherein, in Formula I, X is a modified monocyclic group which is pyrrolidine or piperidine substituted with (CR_{b}R_{b})ₙZ; and

compounds of Formula I wherein, in Formula I, X is a modified monocyclic group which is piperidine substituted with (CR_{b}R_{b})ₙZ where R_{b} is hydrogen and n is 1.

Examples of particular compounds of the invention include:

2-(2,4-Dichlorophenyl)-3,6-diethyl-5-[(2R)-2-(methoxymethyl)pyrrolidin-1-yl]pyrazine;

2-(2-Chloro-4-methoxyphenyl)-3,6-diethyl-5-[(2R)-2-(methoxymethyl)pyrrolidin-1-yl]pyrazine;

2-(2,4-dichlorophenyl)-3,6-diethyl-5-[(2S)-2-(methoxymethyl)pyrrolidin-1-yl]pyrazine;

2-(2-chloro-4-methoxyphenyl)-3,6-diethyl-5-[(2S)-2-(methoxymethyl)pyrrolidin-1-yl]pyrazine;

2-(2-chloro-4-methoxyphenyl)-3,6-diethyl-5-[(3R)-3-(methoxymethyl)pyrrolidin-1-yl]pyrazine;

2-(2-chloro-4-methoxyphenyl)-5-[(3R)-3-(ethoxymethyl)pyrrolidin-1-yl]-3,6-diethylpyrazine;

2-(2-chloro-4-methoxyphenyl)-3,6-diethyl-5-[(3S)-3-(methoxymethyl)pyrrolidin-1-yl]pyrazine;

2-(2-chloro-4-methoxyphenyl)-5-[(3S)-3-(ethoxymethyl)pyrrolidin-1-yl]-3,6-diethylpyrazine; and

2-(2-chloro-4-methoxyphenyl)-3,6-diethyl-5-[4-(methoxymethyl)piperidin-1-yl]pyrazine.

Compounds provided herein can have one or more asymmetric centers or planes, and all chiral (enantiomeric and diastereomeric) and racemic forms of the compound are included in the present invention. Compounds of the invention are isolated in either the racemic form, or in the optically pure form, for example, by resolution of the racemic form by conventional methods such as crystallization in the to presence of a resolving agent, or chromatography, using, for example, a chiral HPLC column, or synthesized by a asymmetric synthesis route enabling the preparation of enantiomerically enriched material. The present invention encompasses all possible tautomers of the compounds represented by Formula (I).

Compounds of the invention can be prepared using the synthetic routes outlined in Chart A. Specific examples of the procedure for the preparation of compounds of the invention are provided in EXAMPLES 5 and 6 below. Starting materials are either commercially available or can be prepared by procedures that would be well known to one of ordinary skill in organic chemistry. As illustrated in Chart A, the pyrazine A-2, for which the point of attachment of X is nitrogen, can be prepared from the suitably functionalized halopyrazine A-I by reaction with a cyclic amine in the presence of a transition metal catalyst (eg, palladium(II) acetate or tris(dibenzylideneacetone)dipalladium(0)), base (eg, sodium or potassium tert-butoxide) in solvents such as but not limited to toluene, DMF, dioxane. (for example see Buchwald, S. L. J. Org. Chem. 2000, 1158.). A variety of cyclic amines are commercially available or can be synthesized by those skilled in the art. The pyrazines A-2, for which the pint of attachment of X is carbon such as an aryl or heteroaryl may be prepared by a transition metal catalyzed coupling reaction and an appropriate metalloaryl reagent such as aryl boronic acids (see for example Miyaura, N.; et al Chem. Rev. 1995, 95, 2457), aryl stannanes (see for example Mitchell, T. N. Synthesis 1992, 803), or aryl Grignards (see for example Miller, J. A. Tetrahedron Lett. 1998, 39, 7275). Halogenation of A-2 can be accomplished by a number of methods well-known to those skilled in the art utilizing reagents such as N-chlorosuccinimide, N-bromosuccinimide, N-iodosuccinimide, bromine, iodine, pyridinium tribromide and trifluoroacetyl hypoiodite in solvents such as dichloromethane, acetic acid, DMF, etc, to give the halopyrazine A-3. Formation of the claimed compounds Formula I is accomplished by a transition metal catalyzed coupling reaction A-3 and an appropriate metalloaryl reagent such as aryl boronic acids, aryl stannanes as described above. Alternatively, A-1 can be coupled with a suitable metalloaryl reagent as described above to provide the arylpyrazine A-4. Oxidation of the sterically less hindered nitrogen can be effected by using a variety of known oxidizing agents (eg, MCPBA, hydrogen peroxide), and the resulting N-oxide can be treated with phosphorous oxychloride to provide the chloropyrazine A-5. Displacement of the chlorine with a cyclic amine, aryl or heteroaryl as described above provides the desired compound.

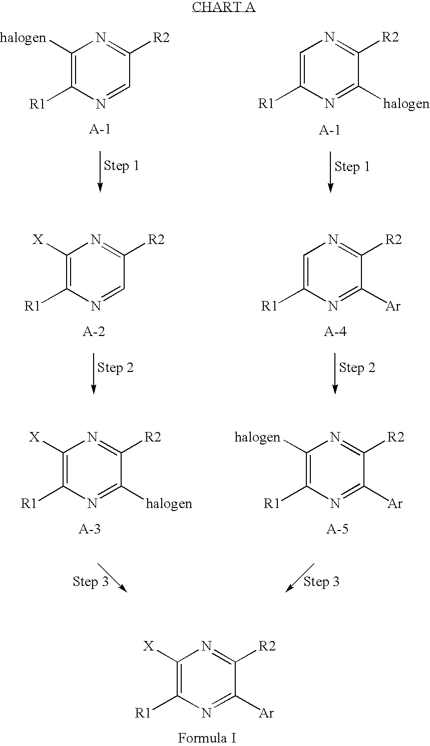

In another aspect, the present invention provides a method of antagonizing a CRF₁ receptor in a mammal, comprising administering to the mammal a therapeutically effective amount of a compound of the invention.

In another aspect, the present invention provides a method of treating a disorder manifesting hypersecretion of CRF in a mammal, comprising administering to the animal a therapeutically effective amount of a compound of the invention.

In another aspect, the present invention provides a method for the treatment of a disorder, the treatment of which can be effected or facilitated by antagonizing CRF₁ receptors, in a mammal, comprising administering to the mammal a therapeutically effective of a compound of the invention.

In another aspect, the present invention provides a method of treating anxiety or depression in a mammal, particularly in a human, comprising administering to the mammal or human a therapeutically effective amount of a compound of the invention.

In another aspect, the present invention provides a method for screening for ligands for CRF₁ receptors, which method comprises: a) carrying out a competitive binding assay with CRF₁ receptors, a compound of the invention which is labelled with a detectable label, and a candidate ligand; and b) determining the ability of said candidate ligand to displace said labelled compound.

In another aspect, the present invention provides a method for detecting CRF receptors in tissue comprising: a) contacting a compound of the invention which is labelled with a detectable label, with a tissue, under conditions that permit binding of the compound to the tissue; and b) detecting the labelled compound bound to the tissue.

In another aspect, the present invention provides a method of inhibiting the binding of CRF to CRF₁ receptors, comprising contacting a compound of the invention with a solution comprising cells expressing the CRF₁ receptors, wherein the compound is present in the solution at a concentration sufficient to inhibit the binding of CRF to the CRF₁ receptors.

In another aspect, the present invention provides an article of manufacture comprising: a) a packaging material; b) a compound of the invention; and c) a label or package insert contained within said packaging material indicating that said compound is effective for treating a pre-selected disorder described herein above.

Compounds of the invention are useful for treating various disorders in a mammal, particularly in a human, such as social anxiety disorder; panic disorder; obsessive-compulsive disorder; anxiety with co-morbid depressive illness; affective disorder; anxiety; depression; irritable bowel syndrome; post-traumatic stress disorder; supranuclear palsy; immune suppression; gastrointestinal disease; anorexia nervosa or other feeding disorder; drug or alcohol withdrawal symptoms; substance abuse disorder (e.g., nicotine, cocaine, ethanol, opiates, or other drugs); inflammatory disorder; fertility problems; disorders the treatment of which can be effected or facilitated by antagonizing CRF, including but not limited to disorders induced or facilitated by CRF; a disorder selected from inflammatory disorders such as rheumatoid arthritis and osteoarthritis, pain, asthma, psoriasis and allergies; generalized anxiety disorder; panic, phobias, obsessive-compulsive disorder; post-traumatic stress disorder; sleep disorders induced by stress; pain perception such as fibromyalgia; mood disorders such as depression, including major depression, single episode depression, recurrent depression, child abuse induced depression, and postpartum depression; dysthemia; bipolar disorders; cyclothymia; fatigue syndrome; stress-induced headache; cancer, human immunodeficiency virus (HIV) infections; neurodegenerative diseases such as Alzheimer's disease, Parkinson's disease and Huntington's disease; gastrointestinal diseases such as ulcers, irritable bowel syndrome, Crohn's disease, spastic colon, diarrhea, and post operative ilius and colonic hypersensitivity associated by psychopathological disturbances or stress; eating disorders such as anorexia and bulimia nervosa; hemorrhagic stress; stress-induced psychotic episodes; euthyroid sick syndrome; syndrome of inappropriate antidiarrhetic hormone (ADH); obesity; infertility; head traumas; spinal cord trauma; ischemic neuronal damage (e.g., cerebral ischemia such as cerebral hippocampal ischemia); excitotoxic neuronal damage; epilepsy; cardiovascular and hear related disorders including hypertension, tachycardia and congestive heart failure; stroke; immune dysfunctions including stress induced immune dysfunctions (e.g., stress induced fevers, porcine stress syndrome, bovine shipping fever, equine paroxysmal fibrillation, and dysfunctions induced by confinement in chickens, sheering stress in sheep or human-animal interaction related stress in dogs); muscular spasms; urinary incontinence; senile dementia of the Alzheimer's type; multiinfarct dementia; amyotrophic lateral sclerosis; chemical dependencies and addictions (e.g., dependences on alcohol, cocaine, heroin, benzodiazepines, or other drugs); osteoporosis; psychosocial dwarfism and hypoglycemia.

Thus, in another aspect, the present invention provides a method of treating a disorder described herein above in a mammal, particularly in a human, comprising administering to the mammal or human a therapeutically effective amount of a compound of the invention.

Particular disorders that can be treated by the method of the invention preferably include the following: affective disorder; anxiety; depression; irritable bowel syndrome; post-traumatic stress disorder; supranuclear palsy; obsessive-compulsive disorder; anxiety with co-morbid depressive illness; Alzheimer's disease; gastrointestinal disease; skin disorders (e.g., acne, psoriasis); anorexia nervosa; social anxiety disorder; bulimia nervosa or other feeding disorder; drug (e.g., dependencies on cocaine, heroin, benzodiazepines, nicotine or other drugs) or alcohol withdrawal symptoms; substance abuse disorder (e.g., nicotine, cocaine, ethanol, opiates, or other drugs); inflammatory disorder; disorders; the treatment of which can be effected or facilitated by antagonizing CRF, including but not limited to disorders induced or facilitated by CRF; or a disorder selected from inflammatory disorders such as rheumatoid arthritis and osteoarthritis, pain, asthma, psoriasis and allergies; generalized anxiety disorder; panic disorder; phobias; obsessive-compulsive disorder; sleep disorders induced by stress; pain perception such as fibromyalgia; mood disorders such as depression, including major depression, single episode depression, recurrent depression, child abuse induced depression, and postpartum depression; dysthymia; bipolar disorders; cyclothymia; fatigue syndrome; stress-induced headache; cancer; neurodegenerative diseases such as, Parkinson's disease and Huntington's disease; gastrointestinal diseases such as ulcers, Crohn's disease, spastic colon, diarrhea, and post operative ilius and colonic hypersensitivity associated by psychopathological disturbances or stress; stress-induced psychotic episodes; syndrome of inappropriate antidiarrhetic hormone (ADH); cardiovascular and hear related disorders including hypertension, tachycardia and congestive heart failure; stroke; senile dementia of the Alzheimer's type; multiinfarct dementia; amyotrophic lateral sclerosis.

Particular disorders that can be treated by the method of the invention more preferably include the following: affective disorder; anxiety; depression generalized anxiety disorder; social anxiety disorder; anxiety; obsessive-compulsive disorder; anxiety with co-morbid depressive illness; panic disorder; mood disorders such as depression, including major depression, single episode depression, recurrent depression, child abuse induced depression, and postpartum depression; bipolar disorders; and post-traumatic stress disorder.

Particular disorders that can be treated by the method of the invention even more preferably include affective disorder, anxiety, and depression.

A compound of this invention can be administered to treat these abnormalities in a mammal or human by means that produce contact of the active agent with the agent's site of action in the body of the mammal or human, oral, topical, parenteral, rectal administration or by inhalation or spray. The term parenteral as used herein includes subcutaneous injections, intravenous, intramuscular, intrasternal injection or infusion techniques. The compounds can be administered by any conventional means available for use in conjunction with pharmaceuticals either as individual therapeutic agent or in combination of therapeutic agents. It can be administered alone, but will generally be administered with a pharmaceutically acceptable carrier selected on the basis of the chosen route of administration and standard pharmaceutical practice. Thus, in another aspect, the invention provides for a pharmaceutical composition comprising a compound of the invention and a pharmaceutically acceptable carrier. One or more compounds of general Formula I may be present in association with one or more non-toxic pharmaceutically acceptable carriers and/or diluents and/or adjuvants and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to 33 provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For C) example, a time delay material such as glyceryl monosterate or glyceryl distearate may be employed.

Formulations for oral use may also be presented as hard gelatin capsules. wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethyleellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring 34 agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension.

This suspension may be formulated according to the known art using those suitable dispersing or wetting agents and suspending agents which have been mentioned above.

The sterile injectable preparation may also be sterile injectable solution or suspension in a non-toxic parentally acceptable diluent or solvent, for example as a solution in 1,3 butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compounds of Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Typical subjects to which compounds of the invention may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like.

Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

For use in the treatment of said diseases or conditions, a compound of this invention can be orally administered at a dosage of the active ingredient of 0.002 to 200 mg/kg of body weight. Ordinarily, a dose of 0.01 to 10 mg/kg in divided doses one to four times a day, or in sustained release formulation will be effective in obtaining the desired pharmacological effect.

Dosage forms (compositions) suitable for administration contain from about 1 mg to about 100 mg of active ingredient per unit. In these pharmaceutical compositions, the active ingredient will ordinarily be present in an amount of about 0.5 to 95% by weight based on the total weight of the composition.

Frequency of dosage may also vary depending on the compound used and the particular disease treated. However, for treatment of most CNS disorders, a dosage regimen of 4 times daily or less is preferred. For the treatment of stress and depression a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy, Preferred compounds of the invention will have certain pharmacological properties. Such properties include, but are not limited to oral bioavailability, low toxicity, low serum protein binding and desirable in vitro and in vivo half-lifes.

Penetration of the blood brain barrier for compounds used to treat CNS disorders is necessary, while low brain levels of compounds used to treat periphereal disorders are often preferred.

The compounds of this invention may also be used as reagents or standards in the biochemical study of neurological function, dysfunction, and disease.

### DEFINITIONS AND CONVENTIONS

The term “substituted aryl” means an aryl group optionally substituted with 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORa;

The term “aryl cycloalkyl” means a bicyclic ring system containing 8 to 14 carbon atoms wherein one ring is aryl and the other ring is fused to the aryl ring and may be fully or partially saturated in the portion of the ring fused to the aryl ring, provided that either ring may act as a point of attachment;

The term “substituted aryl cycloalkyl” means an aryl cycloalkyl group having 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ;

The term “heteroaryl cycloalkyl” means a bicyclic ring system containing 8 to 14 atoms, wherein one ring is heteroaryl and the other ring is fused to the heteroaryl ring and may be fully or partially saturated in the portion of the ring fused to the heteroaryl ring, provided that either ring may act as a point of attachment;

The term “substituted heteroaryl cycloalkyl” means a heteroaryl cycloalkyl group having 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ;

The term “aryl heterocycloalkyl” means a bicyclic ring system containing 8 to 14 atoms, wherein one ring is aryl and the other ring is heterocycloalkyl, provided that either ring may act as a point of attachment;

The term “substituted aryl heterocycloalkyl” means an aryl heterocycloalkyl group having 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ;

The term “heteroaryl heterocycloalkyl” means a bicyclic ring system containing 8 to 14 atoms, wherein one ring is heteroaryl and the other ring is heterocycloalkyl, provided that either ring may act as a point of attachment;

The term “substituted heteroaryl heterocycloalkyl” means an heteroaryl heterocycloalkyl group having 1-5 substituents independently selected from halogen, —NO₂, —CN, —Rₐ, —ORₐ, —S(O)ₘRₐ, —NRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ;

The term “heteroaryl” means a radical attached via a ring carbon or nitrogen atom of a monocyclic aromatic ring containing five or six ring atoms consisting of carbon and 1, 2, 3, or 4 heteroatoms each selected from the group consisting of non-peroxide O, S, N, with appropriate bonding to satisfy valence requirements as well as a radical (attachment at either carbon or nitrogen) of a fused bicyclic heteroaromatic of about eight to ten ring atoms, and includes radicals such as thienyl, benzothienyl, pyridyl, thiazolyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, and benzoxazolyl, pyrazolyl, triazolyl, tetrazolyl, isoxazolyl, oxazolyl, pyrrolyl, isoquinolinyl, cinnolinyl, indazolyl, indolizinyl, phthalazinyl, pydridazinyl, triazinyl, isoindolyl, purinyl, oxadiazolyl, furazanyl, benzofurazanyl, benzothiophenyl, benzothiazolyl, quinazolinyl, quinoxalinyl, naphthridinyl, and furopyridinyl;

The term “substituted heteroaryl” means a heteroaryl group having 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ

The term “heterocycloalkyl”, unless otherwise specified, means a 4 to 8 membered monocylic ring or bicyclic ring, wherein at least one carbon atom is replaced with a heteromember selected from oxygen, nitrogen, —NH—, or —S(O)ₘ— wherein m is zero, 1, or 2, optionally containing from one to three double bonds, provided that the molecule is not aromatic; and provided that ring attachment can occur at either a carbon or nitrogen atom; Heterocycloalkyl includes tetrahydrofuranyl, tetrahydropyranyl, morpholinyl, pyrrolidinyl, piperidinyl, piperazinyl, [2.2.1]-azabicyclic rings, [2.2.2]-azabicyclic rings, [3.3.1]-azabicyclic rings, quinuclidinyl, azetidinyl, azetidinonyl, oxindolyl, dihydroimidazolyl, and pyrrolidinonyl The term “substituted heterocycloalkyl” is a heterocycloalkyl group having 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ;

The term “cycloalkyl” means a monocyclic or bicyclic alkyl moiety, having from 3-10 carbon atoms optionally containing 1 to 2 double bonds provided that the moiety is not aromatic;

The term “substituted cycloalkyl” means an cycloalkyl group having 1-5 substituents independently selected from halogen, —NO₂, —CN, —Rₐ, —ORₐ, —S(O)ₘRₐ, —NRₐRₐ, —C(O)NRₐRₐ, —C(S)NRₐRₐ—S(O)ₘNRₐRₐ, —NRₐS(O)ₘRₐ, —NRₐC(O)ORₐ, —OC(O)NRₐRₐ, —NRₐC(O)NRₐRₐ, —NRₐC(S)NRₐRₐ, —C(O)ORₐ, —C(S)ORₐ, and —OC(O)ORₐ;

Halogen is a group selected from —F, —Cl, —Br, —I;

The term “alkyl” means both straight and branched chain moieties having from 1-10 carbon atoms optionally containing one or more double or triple bonds;

The term “haloalkyl” means an alkyl moiety having from 1-10 carbon atoms and having 1 to (2v+1) independently selected halogen substituent(s) where v is the number of carbon atoms in the moiety;

The term “pharmaceutically acceptable salt” refers to a salt prepared from pharmaceutically acceptable non-toxic acids, including inorganic acids and organic acids. Suitable non-toxic acids include inorganic and organic acids of basic residues such as amines, for example, acetic, benzenesulfonic, benzoic, amphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, barbaric acid, p-toluenesulfonic and the like; and alkali or organic salts of acidic residues such as carboxylic acids, for example, alkali and alkaline earth metal salts derived from the following bases: sodium hydride, sodium hydroxide, potassium hydroxide, calcium hydroxide, aluminum hydroxide, lithium hydroxide, magnesium hydroxide, zinc hydroxide, ammonia, trimethylammonia, triethylammonia, ethylenediamine, lysine, arginine, ornithine, choline, N,N′-dibenzylethylenediamine, chloroprocaine, diethanolamine, procaine, n-benzylphenethylamine, diethylamine, piperazine, tris(hydroxymethyl)-aminomethane, tetramethylammonium hydroxide, and the like. Pharmaceutically acceptable salts of the compounds of the invention can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ea., Mack Publishing Company, Easton, Pa., 1985, p. 1418, the disclosure of which is hereby incorporated by reference.

The term “prodrug” as used herein means any covalently bonded carrier which releases the active parent drug of Formula I in vivo when such prodrug is administered to a mammalian subject. Prodrugs of the compounds of Formula I are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” means compounds that are rapidly transformed in viva to yield the parent compound of formula I, for example by hydrolysis in blood. Functional groups which may be rapidly transformed, by metabolic cleavage, in viva form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in viva, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ea., Elsevier, 1985; Methods in Enzymology, K. Widder et al, Ed., Academic Press, 42, p.309-396, 25 1985; A Textbook of Drug Design and Development, Krogsgaard-Larsen and H. Bundgaard, ea., Chapter 5; “Design and Applications of Prodrugs” p. 113-191, 1991; Advanced Drug Delivery Reviews, H. Bundgard, 8, p. 1-38, 1992; Journal of Pharmaceutical Sciences, 77, p. 285, 30 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ea., American Pharmaceutical Association and Pergamon Press, 1987, which are incorporated herein by reference. “Prodrugs” are considered to be any covalently bonded carriers which release the active parent drug of Formula I in vivo when such prodrug is administered to a mammalian subject. Prodrugs of the compounds of Formula I are prepared by modifying functional groups present in the compounds in such a way that the modifications are cleaved, either in routine manipulation or in vivo, to the parent compounds.

Prodrugs include compounds wherein hydroxy, amine, or sulfhydryl groups are bonded to any group that, when administered to a mammalian subject, cleaves to form a free hydroxyl, amino, or sulfhydryl group, respectively. Examples of Prodrugs include, but are not limited to, acetate, formate and benzoate derivatives of alcohol and amine functional groups in the compounds of Formula I, and the like.

The term “therapeutically effective amount” of a compound of this invention means an amount effective to antagonize abnormal level of CRF or treat the symptoms of affective disorder, anxiety, depression, or other disorders described herein above, in a host.

The term “compound of the invention” means a compound of Formula I, a stereoisomer thereof, a pharmaceutically acceptable salt thereof, or a prodrug thereof.

### EXAMPLES

The following examples are provided to describe the invention in further detail, and intended to illustrate the invention and not to limit the invention in any way.

### Example 1

### CRF1 Receptor Binding Assay for the Evaluation of Biological Activity

The following is a description of the isolation of rat brain membranes for use in the standard binding assay as well as a description of the binding assay itself. It is based on a modified protocol described by De Souza (De Souza, 1987).

To prepare brain membranes for binding assays, rat frontal cortex is homogenized in 10 mL of ice cold tissue buffer (50 mM HEPES buffer pH 7.0, containing 10 mM MgCl₂, 2 mM EGTA, 1 μg/ml aprotinin, 1 μg/ml leupeptin and 1 μg/ml pepstatin). The homogenate is centrifuged at 48,000×g for 10 min. and the resulting pellet rehomogenized in 10 mL of tissue buffer. Following an additional centrifugation at 48,000×g for 10 min., the pellet is resuspended to a protein concentration of 300 μg/mL.

Binding assays are performed in 96 well plates at a final volume of 300 μL. The assays are initiated by the addition of 150 μL membrane suspension to 150 μL of assay buffer containing ¹²⁵I-ovine-CRF (final concentration 150 pM) and various concentrations of inhibitors. The assay buffer is the same as described above for membrane preparation with the addition of 0.1% ovalbumin and 0.15 mM bacitracin. Radioligand binding is terminated after 2 hours at room temperature by filtration through Packard GF/C unifilter plates (presoaked with 0.3% polyethyleneimine) using a Packard cell harvestor. Filters are washed three times with ice cold phosphate buffered saline pH 7.0 containing 0.01% Triton X-100. Filters are assessed for radioactivity in a Packard TopCount. Nonspecific binding is determined in the presence of excess (10 μM) α-helical CRF.

Alternatively, tissues and cells that naturally express CRF receptors, such as IMR-32 human neuroblastoma cells (ATCC; Hogg et al., 1996), can be employed in binding assays analogous to those described above.

IC₅₀ values are calculated using standard methods known in the art, such as with the non-linear curve fitting program RS/1 (BBN Software Products Corp., Cambridge, Mass.). A compound is considered to be active if it has an IC₅₀ value of less than about 10 micromolar (μM) for the inhibition of CRF₁ receptors. The binding affinity of the compounds of Formula I expressed as IC₅₀ values generally ranges from about 0.5 nanomolar to about 10 micromolar. Preferred compounds of Formula I exhibit IC₅₀ of 1 micromolar or less, more preferred compounds of Formula I exhibit IC₅₀ of less than 100 nanomolar or less, still more preferred compounds of Formula I exhibit IC₅₀ of less than 10 nanomolar or less.

### Example 2

### Ex vivo CRF1 Receptor Binding Assay for the Evaluation of Biological Activity

The following is a description of the ex vivo CRF1 receptor binding assay for assessing the biological activity of the compounds of the inventions.

Animal Dosing: Fasted, male, Harlen-bred, Sprague-Dawley rats (170-210 g) were orally dosed with test compound or vehicle, via gastric lavage between 12:30 and 2:00 PM. Compounds were prepared in vehicle (usually 10% soybean oil, 5% polysorbate 80, in dH20). Two hours after drug administration, rats were sacrificed by decapitation, frontal cortices were quickly dissected and placed on dry ice, then frozen at −80° C. until assayed; trunk blood was collected in heparinized tubes, plasma separated by centrifugation (2500 RPM's for 20 minutes), and frozen at −20° C.

Binding Assay: On the day of the assay, tissue samples were weighed and allowed to thaw in ice cold 50 mM Hepes buffer (containing 10 mM MgCl₂, 2 mM EGTA, 1 μg/ml aprotinin, 1 μg/ml leupeptin hemisulfate, and 1 μg/ml pepstatin A, 0.15 mM bacitracin, and 0.1% ovalalbumin, pH=7.0 at 23° C.) and then homogenized for 30 sec at setting 5 (Polytron by Kinematica). Homogenates were incubated (two hours, 23° C., in the dark) with [¹²⁵I] CRF (0.15 nM, NEN) in the presence of assay buffer (as described above) or DMP-904 (10 uM). The assay was terminated by filtration (Packard FilterMate, GF/C filter plates); plates were counted in Packard TopCount LSC; total and non-specific fmoles calculated from DPM's. Data are expressed as % of vehicle controls (specific fmoles bound). Statistical significance was determined using student's t-test.

### Example 3

### Inhibition of CRF Stimulated Adenylate Cyclase Activity

Activities of compounds of the invention can be assessed by assays on the inhibition of CRF-stimulated adenylate cyclase activity. Inhibition of CRF-stimulated adenylate cyclase activity can be performed as previously described [G. Battaglia et al., Synapse 1:572 (1987)]. Briefly, assays are carried out at 37° C. for 10 min in 200 mL of buffer containing 100 mM Tris-HCl (pH 7.4 at 37° C.), 10 mM MgCl₂, 0.4 mM EGTA, 0.1% BSA, 1 mM isobutylmethylxanthine (IBMX), 250 units/mL phosphocreatine kinase, 5 mM creatine phosphate, 100 mM guanosine 5′-triphosphate, 100 nM o-CRF₁ antagonist peptides (various concentrations) and 0.8 mg original wet weight tissue (approximately 40-60 mg protein). Reactions are initiated by the addition of 1 mM ATP/[³²P]ATP (approximately 2-4 mCi/tube) and terminated by the addition of 100 mL of 50 mM Tris-HCl, 45 mM ATP and 2% sodium dodecyl sulfate. In order to monitor the recovery of cAMP, 1 mL of [³H]cAMP (approximately 40,000 dpm) is added to each tube prior to separation. The separation of [³² P]cAMP from [³²P]ATP is performed by sequential elution over Dowex and alumina columns.

Alternatively, adenylate cyclase activity can be assessed in a 96-well format utilizing the Adenylyl Cyclase Activation FlashPlate Assay from NEN Life Sciences according to the protocols provided. Briefly, a fixed amount of radiolabeled cAMP is added to 96-well plates that are precoated with anti-cyclic AMP antibody. Cells or tissues are added and stimulated in the presence or absence of inhibitors. Unlabeled cAMP produced by the cells will displace the radiolabeled cAMP from the antibody. The bound radiolabeled cAMP produces a light signal that can be detected using a microplate scintillation counter such as the Packard TopCount. Increasing amounts of unlabeled cAMP results in a decrease of detectable signal over a set incubation time (2-24 hours).

### Example 4

### in vivo Biological Assay

The in vivo activity of a compound of the present invention can be assessed using any one of the biological assays available and accepted within the art. Illustrative of these tests include the Acoustic Startle Assay, the Stair Climbing Test, and the Chronic Administration Assay. These and other models useful for the testing of compounds of the present invention have been outlined in C. W. Berridge and A. J. Dunn Brain Research Reviews 15:71 (1990). A compound may be tested in any species of rodent or other small mammals.

### Example 5

### Preparation of 2-(2,4-Dichlorophenyl)-3,6-diethyl-5-[(2R)-2-(methoxymethyl)pyrrolidin-1-yl]pyrazine;

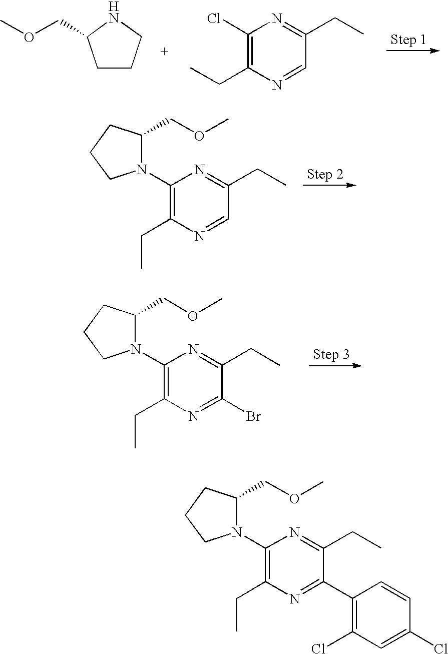

Step 1; 2,5-Diethyl-3-[(2R)-2-(methoxymethyl)pyrrolidin-1-yl]pyrazine. A 20 ml telflon capped vial was charged with 3-chloro-2,5-diethylpyrazine (0.533 g, 3.13 mmol) and (R)-2-(methoxymethyl)pyrrolidine (0.300 g, 2.60 mmol) and 3 ml toluene under N₂. Pd₂(dba)₃ (0.095 g, 0.104 mmol), 2-dicyclohexylphosphino-2′-(N,N-dimethylamino)-biphenyl (0.082 g, 0.208 mmol), and NaOtBu (0.349 g, 3.64 mmol) were added and the solution was heated for 18 h at 95° C. The reaction mixture was partitioned between saturated aqueous NaHCO₃ and CH₂Cl₂ and separated. The combined organic layers were dried with MgSO₄, filtered, and concentrated to give a residue, which was purified by flash chromatography (1/3: EtOAc/heptane) to provide 0.544 g (85%) of 2,5-diethyl-3-[(2R)-2 (methoxymethyl)pyrrolidin-1-yl]pyrazine as an oil. ¹H NMR (400 MHz, CDCl₃) δ 7.80 (s, 1 H), 4.59 (m, 1 H), 3.73 (m, 1 H), 3.60 (dd, 1 H), 3.28 (s, 3 H), 3.26 (m, 2 H), 2.83 (q, 2 H), 2.67 (q, 2 H), 2.17 (m, 1 H), 1.99 (m, 1 H), 1.88 (m, 1 H), 1.27 (m, 6 H);MS (ESI+) for C₁₄H₂₃N₃O m/z 250.1912 (M+H)⁺.

Step 2; 2-Bromo-3,6-diethyl-5-[(2R)-2-(methoxymethyl)pyrrolidin-1-yl]pyrazine. A solution of 2,5-diethyl-3-[(2R)-2 (methoxymethyl)pyrrolidin-1-yl]pyrazine (0.400 g, 1.6 mmol), NBS (0.314 g, 1.76 mmol), and CH₂Cl₂ (10 ml) was stirred at 0° C. for 16 h. The mixture was diluted with Et₂O and separated. The organic layer was washed with NaHCO₃, brine and dried with MgSO₄. The mixture was filtered and concentrated to give a residue that was purified by flash chromatography (1/1: EtOAc/heptane) to give bromo-3,6-diethyl-5-[(2R)-2-(methoxymethyl)pyrrolidin-1-yl]pyrazine as a yellow oil (0.289 g, 56%). ¹H NMR (400 MHz, CDCl₃) δ 4.52 (m, 1 H), 3.74 (m, 1 H), 3.58 (dd, 1 H), 3.35 (s, 3 H), 3.30 (m, 2 H), 2.82 (m, 4 H), 2.15 (m, 1 H), 1.96 (m, 1 H), 1.88 (m, 2 H), 1.28 (m, 6 H); MS (ESI+) for C₁₄H₂₂BrN₃O m/z 328.1020 (M+H)⁺.

Step3; 2-(2,4-Dichlorophenyl)-3,6-diethyl-5-[(2R)-2-(methoxymethyl)pyrrolidin-1-yl]pyrazine,

A mixture of bromo-3,6-diethyl-5-[(2R)-2-(methoxymethyl)pyrrolidin-1-yl]pyrazine (0.12 g, 0.365 mmol), (2,4-dichlorophenyl)-boronic acid (0.0936 g, 0.492 mmol), PdCl₂(PPh₃)₂ (0.032 g, 0.046 mmol), 2 N Na₂CO₃ (0.44 ml), and benzene (2.2 ml) heated at reflux for 16 h. The reaction mixture was diluted with EtOAc and saturated aqueous NaHCO₃ and separated. The aqueous layer was extracted with EtOAc and the combined organic layers were dried with MgSO₄, filtered and concentrated to give a residue that was purified by reverse phase prep HPLC to yield an oil (5 mg, 3%). ¹H NMR (400 MHz, CDCl₃) δ 7.50 (s, 1 H), 7.33 (s, 1 H), 4.64 (m, 1 H), 3.81 (q, 1 H), 3.71 (dd, 1 H), 3.39 (s, 3 H), 3.37 (m, 2 H), 2.89 (q, 2 H), 2.50 (m, 2 H), 2.07 (m, 2 H), 1.95 (m, 2 H), 1.28 (t, 3 H), 1.18 (t, 3 H); MS (ESI+) for C₂₀H₂₅Cl₂N₃O m/z 394.0 (M+H)⁺.

### Example 6

### Preparations of Other Representative Compounds of the Inveniton

The following compounds can be prepared in a similar manner as described in EXAMPLE 5 above:

2-(2-Chloro-4-methoxyphenyl)-3,6-diethyl-5-[(2R)-2-(methoxymethyl)pyrrolidin-1-yl]pyrazine.

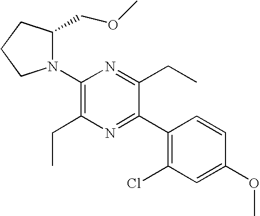

¹H NMR (400 MHz, CDCl₃) δ 7.28 (br s, 1 H), 7.02 (s, 1 H), 6.91 (d, 1 H), 4.64 (m, 1 H), 3.86 (s, 3 H), 3.76 (q, 1 H), 3.78 (dd, 1 H), 3.39 (s, 3 H), 3.35 (m, 2 H), 2.87 (q, 2 H), 2.40-2.65 (m, 2 H), 2.20 (m, 1 H), 2.03 (m, I H), 1.96 (m, 2 H), 1.26 (t, 3 H), 1.17 (t, 3 H); MS (ESI+) for C₂₁H₂₈ClN₃O₂ m/z 390.0 (M+H)⁺.

2-(2,4-dichlorophenyl)-3,6-diethyl-5-[(2S)-2-(methoxymethyl)pyrrolidin-1-yl]pyrazine.

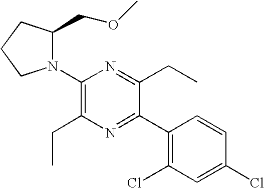

MS (ESI+) for C₂₀H₂₅Cl₂N₃O m/z 394.1440 (M+H)⁺.

2-(2-chloro-4-methoxyphenyl)-3,6-diethyl-5-[(2S)-2-(methoxymethyl)pyrrolidin-1-yl]pyrazine.

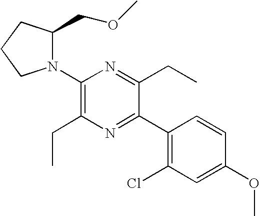

MS (ESI+) for C₂₁H₂₈ClN₃O₂ m/z 390.1952 (M+H)⁺.

2-(2-chloro-4-methoxyphenyl)-3,6-diethyl-5-[(3R)-3-(methoxymethyl)pyrrolidin-1-yl]pyrazine

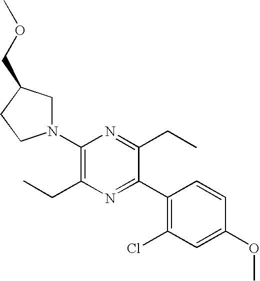

2-(2-chloro-4-methoxyphenyl)-5-[(3R)-3-(ethoxymethyl)pyrrolidin-1-yl]-3,6-diethylpyrazine

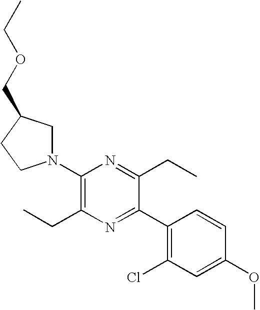

2-(2-chloro-4-methoxyphenyl)-3,6-diethyl-5-[(3S)-3-(methoxymethyl)pyrrolidin-1-yl]pyrazine

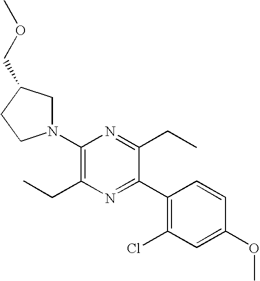

2-(2-chloro-4-methoxyphenyl)-5-[(3S)-3-(ethoxymethylpy ffolidin-1-yl]-3,6-diethylpyrazine

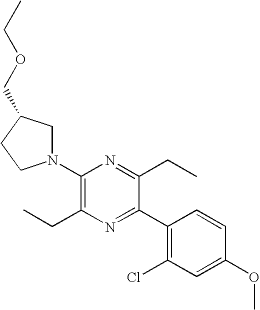

2-(2-chloro-4-methoxyphenyl)-3,6-diethyl-5-[4-(methoxymethyl)piperidin-1-yl]pyrazine

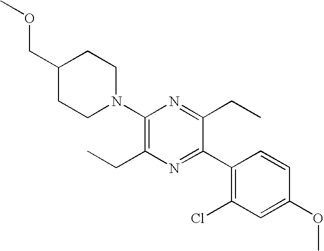

Although the present invention has been described and exemplified in terms of certain embodiments, other embodiments will be apparent to those skilled in the art. The invention is, therefore, not limited to the particular embodiments described and exemplified, but is capable of modification or variation without departing from the spirit of the invention, the full scope of which is delineated by the appended claims.

What is claimed is:

1. A compound of Formula (I) 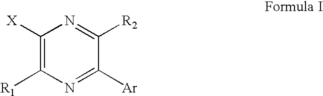
or stereoisomeric forms thereof, or mixtures of stereoisomeric forms thereof, pharmaceutically acceptable prodrugs thereof, or pharmaceutically acceptable salt forms, wherein in formula I,
X is selected from a modified monocyclic group, aryl cycloalkyl, substituted aryl cycloalkyl, heteroaryl cycloalkyl, substituted heteroaryl cycloalkyl, aryl heterocycloalkyl, substituted aryl heterocycloalkyl, heteroaryl heterocycloalkyl, or substituted heteroaryl heterocycloalkyl (point of attachment being either nitrogen or carbon);
modified monocyclic group is selected from cycloalkyl, aryl, heterocycloalkyl, heteroaryl that is substituted with Y or (CR_{b}R_{b})ₙZ, wherein,
Y is selected from CN, NO₂, C(O)Rₐ, C(S)Rₐ, C(O)ORₐ, C(S)ORₐ, C(O)NRₐRₐ, C(S)NRₐRₐ, NRₐC(O)Rₐ, NRₐC(S)Rₐ, NRₐC(O)NRₐRₐ, NRₐC(S)NRₐRₐ, NRₐC(O)ORₐ, OC(O)Rₐ, OC(S)Rₐ, OC(O)NRₐRₐ, OC(S)NRₐRₐ, S(O)ₘNRₐRₐ, NRₐS(O)ₘRₐ, aryl, substituted aryl, heteroaryl, substituted heteroaryl, heterocycloalkyl, substituted heterocycloalkyl, cycloalkyl, substituted cycloalkyl, OR_{c}, and NHR_{c};
Z is selected from Y, ORₐ, NRₐRₐ, and S(O)ₘRₐ;
R_{b} is independently selected from H, alkyl, aryl, heteroaryl, heterocycloalkyl, or cycloalkyl optionally substituted with 1-5 Rt;
R_{c} is selected from aryl, heteroaryl, heterocycloalkyl, or cycloalkyl optionally substituted with 1 to 5 of Rt;
n is selected from 1-6; and
m is selected from 0, 1, and 2;
Ar is selected from aryl, substituted aryl, heteroaryl, substituted heteroaryl;
R₁, R₂, are independently selected from H, halogen, —NO₂, —CN, —ORₐ, —NRₐRₐ, —C(O)Rₐ, —C(O)NRₐRₐ, —C(S)NRₐRₐ, —C(O)ORₐ, —C(S)ORₐ, S(O)ₘRₐ, —S(O)ₘNRₐRₐ, —NRₐS(O)ₘRₐ, —NRₐC(O)ORₐ, —NRₐC(O)Rₐ, —NRₐC(O)NRₐRₐ, —NRₐC(S)NRₐRₐ, and —OC(O)NRₐRₐ, —OC(O)Rₐ, OC(O)ORₐ, CR_{b}R_{b}Z, R_{f};
Rₐ is independently selected from H, alkyl, cycloalkyl, haloalkyl, aryl, heteroaryl, or heterocycloalkyl optionally substituted with 1 to 5 of Rₜ, oxo (═O), thione (═S), phenyl, heteroaryl, or heterocycloalkyl where phenyl, heteroaryl, and heterocycloalkyl are optionally substituted with 1 to 5 independently taken from Rₜ;
R_{f} is independently selected from ethyl, propyl, butyl, pentyl, cycloalkyl, haloalkyl, aryl, heteroaryl, or heterocycloalkyl optionally substituted with 1 to 5 of Rₜ, oxo (═O), thione (═S), phenyl, heteroaryl, or heterocycloalkyl where phenyl, heteroaryl, and heterocycloalkyl are optionally substituted with 1 to 5 independently taken from Rₜ;
Rₜ is independently selected from R_{w}, halogen, —NO₂, —NR_{w}R_{w}, —OR_{w}, —SR_{w}, —CN, —C(O)NR_{w}R_{w}, —C(O)R_{w}, —OC(O)NR_{w}R_{w}, —OC(O)R_{w}, —NR_{w}C(O)R_{w}, —NR_{w}C(O)NR_{w}R_{w}, —NR_{w}C(O)OR_{w}, —S(O)ₘR_{w}R_{w}, —NR_{w}S(O)ₘR_{w}, —S(O)₂NR_{w}R_{w}, —NR_{w}S(O)₂NR_{w}R_{w}; and
R_{w} is independently selected from H, alkyl, cycloalkyl, phenyl, benzyl, heteroaryl or heterocycle where phenyl, benzyl, heteroaryl and heterocycloalkyl may be optionally substituted with alkyl or halogen.

2. A compound according to claim 1 wherein, in Formula I, X is a modified monocyclic group.

3. A compound according to claim 2 wherein the modified monocyclic group is pyrrolidine or piperidine substituted with (CR_{b}R_{b})ₙZ.

4. A compound according to claim 3 wherein the modified monocyclic group is piperidine substituted with (CR_{b}R_{b})ₙZ where R_{b} is hydrogen and n is 1.

5. A compound according to claim 1, which is
2-(2,4-Dichlorophenyl)-3,6-diethyl-5-[(2R)-2-(methoxymethyl)pyrrolidin-1-yl]pyrazine;
2-(2-Chloro-4-methoxyphenyl)-3,6-diethyl-5-[(2R)-2-(methoxymethyl)pyrrolidin-1-yl]pyrazine;
2-(2,4-dichlorophenyl)-3,6-diethyl-5-[(2S)-2-(methoxymethyl)pyrrolidin-1-yl]pyrazine;
2-(2-chloro-4-methoxyphenyl)-3,6-diethyl-5-[(2S)-2-(methoxymethyl)pyrrolidin-1-yl]pyrazine;
2-(2-chloro-4-methoxyphenyl)-3,6-diethyl-5-[(3R)-3-(methoxymethyl)pyrrolidin-1-yl]pyrazine;
2-(2-chloro-4-methoxyphenyl)-5-[(3R)-3-(ethoxymethyl)pyrrolidin-1-yl]-3,6-diethylpyrazine;
2-(2-chloro-4-methoxyphenyl)-3,6-diethyl-5-[(3S)-3-(methoxymethyl)pyrrolidin-1-yl]pyrazine;
2-(2-chloro-4-methoxyphenyl)-5-[(3S)-3-(ethoxymethyl)pyrrolidin-1-yl]-3,6-diethylpyrazine;
2-(2-chloro-4-methoxyphenyl)-3,6-diethyl-5-[4-(methoxymethyl)piperidin-1-yl]pyrazine, or
a pharmaceutically acceptable salt of any said compound.

6. A pharmaceutical composition comprising a pharmaceutically acceptable carrier and a therapeutically effective amount of a compound of claim 1.

7. A method of antagonizing a CRF receptor in a mammal, comprising administering to the mammal, a therapeutically effective amount of a compound of claim 1.

8. A method of treating a disorder manifesting hypersecretion of CRF in a warm-blooded animal, comprising administering to the animal a therapeutically effective amount of a compound of claim 1.

9. A method for the treatment of a disorder, the treatment of which can be effected or facilitated by antagonizing CRF₁ comprising administering to the mammal a therapeutically effective amount of a compound of compound of claim 1.

10. A method for screening for ligands for CRF receptors, which method comprises:
a) carrying out a competitive binding assay with a CRF receptor, a compound of claim 1, which is labelled with a detectable label, and a candidate ligand; and b) determining the ability of said candidate ligand to displace said labelled compound.

11. A method for detecting CRF receptors in tissue comprising: a) contacting a compound of claim 1, which is labelled with a detectable label, with a tissue, under conditions that permit binding of the compound to the tissue; and b) detecting the labelled compound bound to the tissue.

12. A method of inhibiting the binding of CRF to a CRF-1 receptor, comprising contacting a compound of claim 1, with cells expressing the CRF1 receptor, wherein the compound is present in the solution at a concentration sufficient to inhibit the binding of CRF to the CRF-1 receptor.

13. The method of claim 12, wherein the cells are IMR32 cells.

14. A compound according to claim 1, wherein the compound exhibits an IC50 for CRF binding of 1 micromolar or less.

15. A compound according to claim 1, wherein the compound exhibits an IC50 for CRF binding of 100 nanomolar or less.

16. A compound according to claim 1, wherein the compound exhibits an IC50 for CRF binding of 10 nanomolar or less in a standard assay of CRF binding.

17. A method of promoting smoking cessation, comprising administering to a patient in need thereof an effective amount of a compound of claim 1.

18. A method of treating a disorder in a human, comprising administering to the human a therapeutically effective amount of a compound of claim 1, wherein the disorder is selected the group consisting of anxiety-related disorders; mood disorders; post-traumatic stress disorder; supranuclear palsy; immune suppression; drug or alcohol withdrawal symptoms; inflammatory disorders; pain; asthma; psoriasis and allergies; phobias; sleep disorders induced by stress; fibromyalgia; dysthemia; bipolar disorders; cyclothymia; fatigue syndrome; stress-induced headache; cancer; human immunodeficiency virus infections; neurodegenerative diseases; gastrointestinal diseases; eating disorders; hemorrhagic stress; stress-induced psychotic episodes; euthyroid sick syndrome; syndrome of inappropriate antidiarrhetic hormone; obesity; infertility; head traumas; spinal cord trauma; ischemic neuronal damage; excitotoxic neuronal damage; epilepsy; cardiovascular and heart related disorders; immune dysfunctions; muscular spasms; urinary incontinence; senile dementia of the Alzheimer's type; multiinfarct dementia; amyotrophic lateral sclerosis; chemical dependencies and addictions; psychosocial dwarfism, hypoglycemia, and skin disorders; and hair loss.

19. A method according to claim 18 wherein the disorder is selected the group consisting of anxiety-related disorders; mood disorders; bipolar disorders; post-traumatic stress disorder; inflammatory disorders; chemical dependencies and addictions; gastrointestinal disorders; and skin disorders.

20. A method according to claim 19 wherein the disorder is selected from anxiety-related disorders and mood.

21. A method according to claim 20 wherein the anxiety-related disorder is generalized anxiety and the mood disorder is depression.
